(19) United States
(12) Patent Application Publication
(10) Pub. No.: US 2004/0097838 A1
(43) Pub. Date: May 20, 2004

(54) System and apparatus for providing quantified hand analysis

(76) Inventors: William C. Paske, Stafford, TX (US); Jeffrey M. Sutherland, Humble, TX (US)

Correspondence Address: FULBRIGHT & JAWORSKI, LLP, 1301 MCKINNEY, SUITE 5100, HOUSTON, TX, 77010-3095, US

(21) Appl. No.: 10/715,175

(22) Filed: Nov. 17, 2003

Related U.S. Application Data
(60) Provisional application No. 60/427,393, filed on Nov. 19, 2002.

(51) Int. Cl. A61B 5/103; A61B 5/117

(52) U.S. Cl. 600/587; 600/595

(57) ABSTRACT

A non-invasive system and method of determining the presence or absence of neural, muscular, soft tissue, bone or joint damage to a person's hand and/or wrist. The system includes a plurality of contact points, one of which is engageable by the person's thumb and two of which are engageable by other digits of the hand, at least one of the other digits being innervated by the ulnar nerve. Force detectors are operatively connected to at least two of the contact points for measuring the quantity of force applied through respective contact points as the thumb and other digits are brought toward each other in a natural gripping motion. The measured forces are displayed for analysis of the hand and/or wrist.

This application claims priority to U.S. Provisional Application Serial No. 60/427,393, filed Nov. 19, 2002.

### TECHNICAL FIELD

The present invention relates to a system, method, and apparatus for measuring both the absolute and relative muscular strength of hand digits innervated by the median and ulnar nerves. These measurements may be used to distinguish between the pattern characteristics of a healthy and injured hand.

### BACKGROUND OF THE INVENTION

Repetitive motion disorders such as carpal tunnel syndrome are the most common occupational illnesses being reported among a wide range of professions which involve significant hand/wrist motions such as typists (particularly forceful typists), meat cutters, etc. Carpal tunnel syndrome (CTS) symptoms include decreased strength; paresthesias (burning or tingling) in the thumb and adjacent fingers; pain in the wrist, palm, forearm; decreased median nerve conduction velocities; and sensory loss in areas of the hand innervated by the median nerve.

Symptoms resembling CTS may be due to chronic diseases such as rheumatoid arthritis or diabetes mellitus; congenital defects; acute trauma; age; birth control pill usage; and pregnancy. Historically, to aid in the diagnosis of the symptoms, Tinel's Sign, Phalen's Test, and Electromyography nerve conduction tests are usually conducted. Additional tests have been developed to aid in symptom diagnosis which are based on specific measurable parameters such as: the expected weakness of the diseased hand; the decreased sensitivity of the fingers to applied vibrational stimulus; restricted range of motion; sensitivity to applied pressure and chemical analysis of body proteins.

By lightly percussing the course of the median nerve for several seconds, a tingling sensation may be felt in the distribution of the median nerve, indicating a positive Tinel's Sign that suggests that CTS may be present. Phalen's Test generally requires the patient to press the backs of both hands together forming right angles, or holding the patient's wrist in acute flexion for 60 seconds. Numbness or tingling developing over the distribution of the median nerve is an indication that CTS is possible. Positive results in these tests are usually followed by electromyography (EMG) nerve conduction studies. An EMG is sensitive enough to detect the syndrome in 85% of those tested. However, a positive test result cannot be achieved in cases where the nerve has not been damaged to the extent that conduction has been impaired. Presently, objective clinical information regarding the structure and functionality of the nervous system may be collected by recording electrical signals generated or propagated through the muscular or neural paths of interest. These electrodiagnostic tests require electrodes to be affixed to the patient under study so that electrical signals passing through the area of study may be collected and recorded. In general, these systems require the use of electrodes for both collection and stimulation, which may cause surface bums and/or pain to the patient during the evaluation. Two safety issues are present when using these tests: patient/practitioner contamination due to blood born infection, and possible electrical shock. Both of these hazards may be minimized through disposable electrodes and through proper electrical shielding to make the systems “touch-proof” (shock-proof).

To significantly augment the present electrodiagnostic methods, a methodology relying on totally different physics is necessary. The prior art, not relying on the art of electrodiagnostics, discloses several devices which measure a variety of parameters which are intended to provide evidence of injury, specifically CTS. These prior devices fall into distinct categories which rely or different physical concepts or address specific symptoms. These groups are:

1. Muscle Strength: Device measures the strength of the finger(s), thumb, or the whole hand grip.

2. Vibration Threshold: Device measures the threshold of the finger's sensitivity to applied vibrations.

3. External Pressure: Device applies an external pressure to the appropriate nerve pathway and then asks for subjective report of numbness or weakness.

4. Body Mobility: Device assesses range of motion in conjunction with EMG testing;

5. Chemical Analysis: Body fluids are extracted and analyzed via electrophoresis or immunoassay to measure relative amounts of proteins which may be used to infer injury.

U.S. Pat. No. 4,774,966 describes a muscle strength measurement. This device is intended to provide an objective method to measure any weakness present in the hand. If present, this weakness might be related to carpal tunnel syndrome and as such, provide an early identification of the problem. This measurement is accomplished by measuring the strength of the intrinsic hand muscles innervated by the median nerve after it passes through the carpal tunnel. The motion of the hand is limited by the fixture provided such that only the muscles supplied by the median nerve are measured. The test provides a screening by comparing the strength measured to a predefined normal strength. If the test individual should indicate weakness, further evaluation would be indicated. Weakness could be due to peripheral neuropathy, cerebral damage, cervical cord damage and neuromuscular disease.

U.S. Pat. No. 5,163,443 describes a muscle strength measurement. This invention provides for the measurement of applied forces exerted by the hand, wrist and forearm with the intention of detecting the presence of cumulative trauma disorders, such as CTS. The forces measured are correlated with forces expected or required in specific work tasks, by comparing maximal strength as well as mobility of the limb in question. In addition, the system will provide a grip and finger strength measurement. These measurements are made by a single transducer.

U.S. Pat. No. 5,471,996 describes a muscle strength measurement. This patent describes an apparatus which allows a measurement of the muscle strength of the thumb to be studied. Assessment of the strength of the abductor pollicis brevis is important in the diagnosis of some types of neck and elbow injuries, and for CTS and other hand disorders. This apparatus restrains the hand and allows restricted movement to guarantee a reproducible force is applied by the thumb. Isometric and isokinetic testing is possible with this apparatus.

U.S. Pat. No. 5,002,065 describes a vibration threshold measurement. Nerve injury may be induced by exposure to excessive vibration, causing the sensory threshold to be increased for the afflicted fingers. It has also been suggested that an increase in the perception threshold for vibration stimuli is the earliest detectable objective sign in patients with CTS. This patent teaches that serious injury caused by exposure to vibration may be prevented by predicting the injury to the hands prior to irreversible damage by observing the patient's threshold to vibrational stimuli. This same method may be used to measure sensory disturbances such as CTS. This invention provides for the application of a normalized vibrational stimulus to a finger of the patient. The frequency and amplitude of the stimulus are discrete and variable. As the test ensues and the frequency/amplitude are increased, the patient is requested to indicate the onset of sensory perception of the stimuli. The patient is also asked to indicate the loss of detection of the stimuli while the frequency/amplitude sweep is decreasing. The patent addresses the conditions of hand position, temperature, and uniform pressure during testing.

U.S. Pat. No. 5,230,345 describes a vibration threshold measurement. This patent provides for a system and method for detecting CTS in a patient by utilizing a vibratory waveform having a discrete frequency and a variable amplitude. The waveform is applied directly to the finger via contact with a speaker cone. A single finger is measured during the test procedure. When the stimuli is detected by the patient, the patient “clicks” the mouse which sends a control signal to the computer. This procedure is completed three times and the results averaged. The results are compared to a predetermined threshold baseline. Measurements exceeding the baseline indicate a diseased condition in the patient.

U.S. Pat. No. 5,301,683 describes an external pressure measurement. Direct pressure is applied for thirty seconds to the median nerve as it passes through the carpal tunnel at base of the wrist. Patients having anatomic evidence of CTS will experience and report numbness, pain, weakness or paresthesias in the distribution of the median nerve distal to the carpel tunnel. This patent provides a system and method for application and control of this applied pressure, so that repeatable tests may be accomplished.

U.S. Pat. No. 5,513,651 describes a body mobility measurement combined with a surface electromyography test. This patent uses a range of motion arm (ROMA) having six degrees of freedom to determine the range of motion in the upper and lower back while recording an exerting force on a strain gauge to simulate lifting conditions. This system will provide a non-invasive, non-loading test for analyzing myofacial injuries and repetitive stress injures. This invention will provide a means to look at the relationship between muscle groups to determine if the problem is cervical, CTS or cubital tunnel.

U.S. Pat. No. 5,583,201 describes a chemical analysis measurement. This invention provides for the diagnosis of peripheral nerve damage, including the damage which causes back and neck pain. A body fluid sample is extracted and subjected to a two-dimensional electrophoresis or an immunoassay. A diagnosis can be made by comparing the relative amounts of protein or proteins which increase or decrease in concentration to a standard control. This study determines whether the pain is caused by muscle or fibrous tissue injury.

The measurements described above relating to the strength of the muscles under study use predetermined standards of what is considered normal. The measurements are also limited to the muscle groups innervated by the median nerve. Since the hand also involves the ulnar nerve (third and small finger), these measurements are not conclusive. Individual strength variations due to relative degree of muscle tone could also mitigate these studies. The measurements relating to vibration sensitivity rely on the patient's compliance to provide an accurate threshold determination. The external pressure measurement described also relies on patient response. The mobility measurements described uses EMG measurements in conjunction with the range of motion tests to remove the patient's subjective involvement. The chemical assay methodology described does not require the patient's compliance, but is invasive.

### SUMMARY OF THE INVENTION

The present invention relates to a system, method, and apparatus for measuring both the absolute and relative muscular strength of hand digits innervated by the median and ulnar nerves. These measurements may be used to distinguish between the pattern characteristics of a healthy and injured hand. One embodiment of the present invention is directed to a system for objective, repeatable, non-invasive analysis of a person's wrist complex comprising a fixture with three or more contact points, wherein the three or more contact points are positioned such that forces applied thereto are balanced and wherein such contact points are configured to minimize the side loads. In one aspect of the described system force detectors are operatively connected to at least two of contact points, one of which is engaged by a digit innervated by the ulnar nerve and the other by a digit innervated by the median nerve, for measuring the force applied by said digits. In another aspect the described system a display unit displays information about the forces. In another aspect of the described system at least some of the contact points are slideably engaged to the fixture. In another aspect of the described system at least one of the contact points is slick. In another aspect of the described system at least one of the contact points is adjustable. In another aspect of the described system at least some of the contact points are made, at least partially, out of a rigid, substantially incompressible material. In another aspect of the described system a display unit displays in a time frequency manner information on the forces applied to the force detectors. In another aspect of the described system a fixture includes contact points for a thumb, index finger, and small finger of a hand, wherein the contact points are engageable by the pads of the fingertips of the thumb and fingers. In another aspect of the described system contact points are slick so that the surface of each contact point engaged by the thumb and fingers is generally perpendicular to the force exerted thereon. In another aspect of the described system contact points are slideably engaged to the fixture so that the surface of each contact point engaged by the thumb and fingers is generally perpendicular to the force exerted thereon. In another aspect of the described system at least some contact points are configured such that the projection of forces applied to the contact points do not intersect. In another aspect of the described system force detectors are operatively connected to the contact points for the thumb, index finger, and small finger. In another aspect of the described system an upper contact point and a lower contact point are configured such that the projection of the force applied to the upper contact point projects away from the lower contact point. In another aspect of the described system a displacement measuring means operatively connected to the three or more contact points for measuring the displacement of said three or more contact points.

Another embodiment includes a non-invasive method of determining the presence or absence of neural, muscular, soft tissue, bone or joint damage to wrist complex comprising the steps of (a) engaging contact points with at least one digit innervated by the ulnar nerve and at least one digit innervated by the median nerve, wherein the contact points are configured to transmit forces normal to their surface; (b) applying force on the contact points with said digits; and (c) measuring the force applied to at least two of said contact points to provide quantifiable outputs therefor, wherein the outputs are used to diagnose wrist complex diseases and injuries. One aspect of the method includes quantifiable outputs that represent the forces applied by at least two digits innervated by different nerves are displayed. In another aspect of the method the outputs are displayed as a function selected from the group consisting of time, frequency, phase, and any combination thereof. In another aspect of the method the measurements are processed by computer for storage or immediate use. In another aspect of the method the diseases and injuries are diagnosed using a technique selected from the group consisting of pattern recognition, neural networks, frequency analysis, phase analysis, signature analysis, graphic displays, and any combination thereof. In another aspect of the method the measurements are compared to earlier measurements at a frequency selected from the group consisting of hourly, daily, weekly, yearly, and any combination thereof to determine long term effects of said diseases or injuries. In another aspect of the method the force is applied to said contact points for a prolonged period of time. In another aspect of the method the force is applied repeatedly to said contact points. In another aspect of the method a visual or audible signal is produced when force should be applied to said contact points. In another aspect of the method the contact points are provided on said fixture that allow displacement measurements to be made. In another aspect of the method the fixture is designed so that the force applied normal to the surface of the contact points is at least 70% of the total force.

Another embodiment includes a force measuring device for transmitting forces applied by a hand comprising a fixture sized to be gripped by a hand; at least two contact points operably attached to said fixture to transmit the force applied normal to the surface of said contact point; a force detector coupled to said contact points for measuring force transmitted to said contact point. In one aspect of the device the fixture comprises an upper surface and a lower surface. In another aspect of the device the upper surface is parallel to the lower surface. In another aspect of the device a contact point is slideably engaged to the upper surface of the fixture. In another aspect of the device a contact point is slideably engaged to said fixture using rollers, wheels, bearings, rails, lubricant or any combination thereof. In another aspect of the device a contact point is slick. In another aspect of the device a contact point is coated with a lubricant. In another aspect of the device the fixture comprises an interior upper surface and an interior lower surface. In another aspect of the device the interior upper surface is parallel to the interior lower surface. In another aspect of the device a contact point is slideably engaged to the interior upper surface of said fixture. In another aspect of the device a contact point is slideably engaged to said fixture using rollers, wheels, bearings, rails, lubricant or any combination thereof. In another aspect of the device a contact point is substantially frictionless to reduce side loads.

Displacement measurements are useful in studying or in determining types of motion responsible for detected injuries.

Other objects and advantages of the invention will be apparent from reading the description which follows in conjunction with the accompanying drawings.

### BRIEF DESCRIPTION OF THE DRAWINGS

FIGS. 1a through 1d are schematics of fixtures used in the non-invasive system and method of the present invention for determining the presence or absence of neural damage to a person's hand and/or wrist, according to a preferred embodiment thereof;

FIG. 2 is a schematic of a contact point on rollers;

FIG. 3 is a block schematic of the disclosed system for measuring force applied by digits as a function of time and wrist position;

FIG. 4a is a plot of force versus elapsed time measurements for the thumb of a person utilizing the system of the present invention, according to a preferred embodiment thereof;

FIG. 4b is a comparative plot of force versus elapsed time measurements-for the small finger of a person utilizing the same system as with FIG. 4a.

FIG. 4c is a plot of force versus pulse representing muscle fatigue of the small finger over a period of time.

FIGS. 5a through 5d is a sequence of plots showing the ratiometric strengths and coordination abilities of the test subject, FIG. 5a representing a quality check by comparing the sum of the finger forces to the opposing thumb force, FIG. 5b representing the data collected for the index and small fingers,

FIG. 5c representing the data collected for the index finger and the thumb and FIG. 5d representing the data collected for the small finger and the thumb; and

FIGS. 6a and 6b are comparison plots of the measured response between the index finger and the small finger for two individuals, FIG. 6a representing applied force and the relatively smooth response of a normal hand, FIG. 6b representing the very erratic behavior in a hand previously diagnosed as suffering from CTS.

FIG. 7 is a diagram of a hand showing finger nomenclature.

### DETAILED DESCRIPTION OF THE INVENTION

The present invention provides a system, method, and apparatus for measuring the forces applied by digits of a hand as a function of time and hand-wrist position (e.g., flexed or neutral). The forces are measured using suitable force detectors such as strain gauges which may be accurately calibrated to provide long term trend studies of the subject hand(s). These detectors are placed on a fixture which is held or supported by the subject, allowing the subject's hand to apply force to the fixture. For example, the subject may be asked to perform a series of finger tip squeeze motions which could range from a single squeeze held as long as the subject is capable to a series of squeeze, release, squeeze, release patterns which may last from a few seconds to several minutes. The forces applied by the subject are then recorded and stored for analysis. Alternatively, the forces may be displayed numerically or graphically for real time analysis.

The present invention provides rapid, non-invasive, reproducible, quantifiable measurements for:

1. Relative and absolute digit strength for the hand(s) under study, which may be compared to tests completed months or years earlier in a statistically significant manner.

2. Muscle fatigue rates so that long term studies will be able to discern improvement or degradation of the hand(s).

3. Relative hand coordination which provides quantified documentation for improvement or degradation of the hand(s).

4. Supporting or defeating claims of muscle or nerve damage related to repetitive motion injury, carpal tunnel injury, trauma, or other neural disorder.

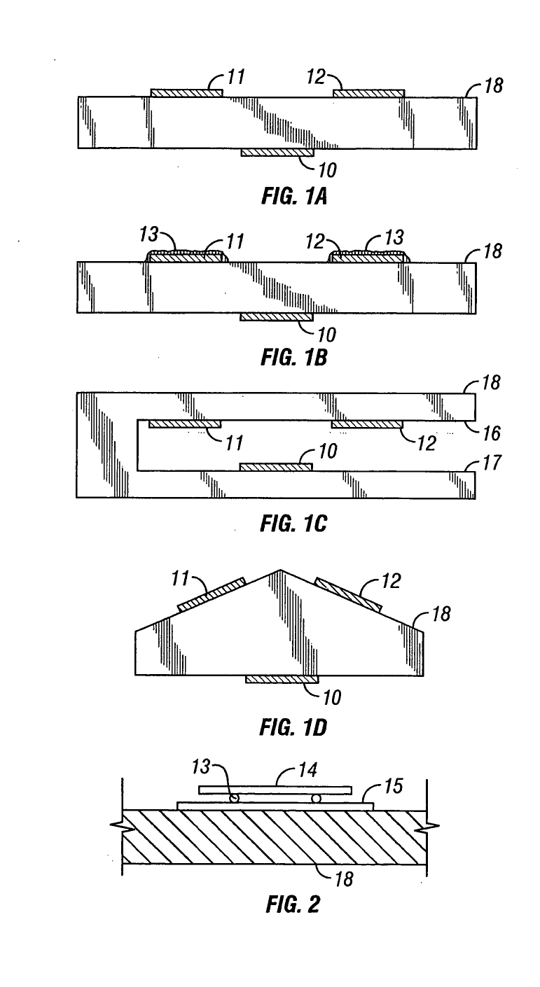

FIGS. 1-6 are provided as illustrations of a system, method, and apparatus for measuring the absolute and relative hand strength and using those measurements to distinguish between healthy and injured hands. Multiple aspects of the invention are embodied in the system, method, and apparatus illustrated herein. However, it should be noted that the detailed description of this preferred system, apparatus, and method is provided to facilitate description of the inventive system, method, and apparatus. It will be apparent to one skilled in the art, upon reading the detailed description and viewing the accompanying drawings, that the invention or certain aspects of the invention is also adapted for use in or with other environments, diagnostic systems, combinations of fingers, and fixture shapes. Accordingly, the present invention is not intended to be limited to the system, method, and apparatus specifically described and illustrated herein.

FIGS. 1a through 1d is a schematic of fixture 18 used to measure forces applied by digits of the hand. Fixture 18 may be adjustable to accommodate more than one hand shape and size. Forces are applied to fixture 18 through contact points 10, 11, and 12. Although three contact points are shown, more than three contact points may be used. For example, a fixture can be configured with ten contact points. A fixture may also have two contact points. The applied forces are measured by strain gauges connected to the fixture in a manner consistent with those schooled in the art of manufacturing loadcells.

A normally functioning hand performs most operations in a relatively smooth, repeatable manner. While the strength of the different digits may vary, the relative functionality between the digits remains smooth and consistent. The smoothness and consistency with which the hand performs these functions can be thought of as coordination.

Contact points 10, 11, and 12 are slick to emphasize the coordination or lack of coordination present in the hand using the apparatus. The component of an applied load that is normal to the surface of a contact point depends in part on the slickness of the contact point. A slick contact point reduces the frictional forces available to support side loads. Thus, if a significant side load is applied to a slick contact point, the finger applying such load will slide across the surface of the contact point. To prevent the slide, the finger will have to apply force in a direction that is substantially normal to the surface of the contact point. Thus, slick contact points minimize the side loads applied to the contact points and result in a substantial portion of the total force being applied normal to the surface of the contact point.

Slick, low friction contact points can be created using polished materials. A slick surface can be created or enhanced by lubricating the existing surface with oil, water, silicone, powder, lotion, or the like. Coating 13 is shown in FIG. 1b. It is readily understood that this list of coatings is not exclusive, and many other materials as well as combinations of materials may be used. As used herein, “slick” refers to a surface configured to be as frictionless as reasonably and/or commercially practical. Coating 13 may be placed on one or more of the contact points, depending on the application.

In an alternative to slick contact points, contact points may be slideably engaged to fixture 18 in a way that allows the contact points to slide over the surface of the fixture. For example, contact points may be slideably engaged to a fixture using rollers, wheels, bearings, rails, or lubrication under the contact points. A configuration using wheels or rollers is shown in FIG. 2. FIG. 2 shows a slide plate 14, wheels 13, contact point 15, and fixture 18.

The contact points may be adapted with springs, air bags or the like to provide measurements of displacement caused by force applied by the hand digits. Displacement measurements are useful for studying or determining types of motion responsible for detected injuries. The contact points may also me made from a substantially incompressible material. One skilled in the art understands that substantially incompressible means material that does not deform under normal finger loads to an extent that impairs the accuracy of the force measurements. One skilled in the art further understands that substantially incompressible also means that the material does not deform under finger loads to an extent that the deformation allows the contacts points to support side loads greater those that would be expected for a given slickness of material.

Contact points can be selected to accept any dynamic range. The dynamic range for each contact point may be the same, different, or some combination of the same and different. For example, contact point 10 may have a dynamic range from 0 to 20 lbs (0-9.1 kgf) and contact points 11 and 12 may have a dynamic range from 0 to 10 lbs (0-4.5 kgf). It is understood by one skilled in the art that the dynamic range of the contact points can be varied according to the application.

In fixture 18 in FIG. 1a, contact point 10 is positioned approximately midway between contact points 11 and 12. This configuration accentuates the balance problems experienced by subjects using the apparatus and as a result, demonstrates a lack of coordination present in an injured hand. It is readily understood by one skilled in the art, however, that the relative location of the contact points can be varied according to the application. For example, fatigue measurements and timing jitter measurements can be made in unsymmetrical configurations, such as when the thumb contact point is not located midway between contact points 11 and 12.

The geometry of FIG. 1a allows the three loadcells to be calibrated at the same time. By placing the fixture on a smooth surface, resting on the two finger contact points, the thumb loadcell may be loaded with known weights of suitable range to calibrate the three loadcells simultaneously. Knowing the geometry of the loadcells allows the calibrator to know how the applied load is balanced by the two contacts points (11 and 12). Typical calibration results using seven calibration weights show linear correlation coefficients for all three loadcells to 0.9999.

FIGS. 1b, 1c, and 1d show alternative configurations of the fixture shown in FIG. 1a. Additionally, one skilled in the art understands that fixtures may be many different shapes, depending on the application. FIG. 1c shows a fixture with interior upper surface 16 and interior lower surface 17. The embodiments disclosed in FIGS. 1a, 1b, 1c, and 1d are not intended to limit the shape of the fixture. Instead, they are intended as representative samples.

Fixture 18 may be shaped to simulate devices normally used by the tested subject; e.g. a mouse, computer pointing device, computer keyboard, etc. This could lead to redesign or altered use of such devices.

The fixture is held by the fingertips of the digits being tested. The fingertips only touch the fixture at the contact points. Held this way, the fixture defines a plane through which the forces must act in order to maintain a grip on the fixture. The fixture should NOT be held by the finger or thumb joints, as this defeats the balance requirement for the measurements. The fixture does not limit the hand-wrist orientation, and this orientation may be varied to aid in diagnosis of any suspected illness or injury. Problems present in one or the other, or both nerve systems will be evident in how the digits balance the fixture during tests. Strength of the test subject's hand is not critical since comparative measurements will be made for all three digits. However, due to the fixture geometry and the slick contact points, the total force applied by the thumb will equal the forces applied by the fingers. This force balance is shown in by equation Eq. (1) below.

F₁₀=F₁₁+F₁₂ Eq.(1)

F₁₀=Force applied to contact point 10

F₁₁=Force applied to contact point 11

F₁₂=Force applied to contact point 12

How the forces are balanced provides information concerning the health of the muscles and nerves involved in the measurement. Forming a balanced force system as described above shows clearly any problems with the median nerve, ulnar nerve, or both nerve paths. The presence or absence of pain also effects the way in which these forces are balanced. Cross plots of all possible pairs of the measured forces provide diagnostics which may also infer hand coordination. The data sample times of the preferred embodiment are fast enough to resolve time increments of 2.0 ms. Data times can be varied to suit the particular application. Poor coordination between any two of the measured digits is demonstrated by very erratic cross plots produced by jerky hand motions. Muscle fatigue can be readily determined by computing the rate of decay or force attenuation as a function of the time the forces are applied. All of these parameters, applied force, coordination, fatigue, etc, may be determined as a function of minutes, hours, days, weeks, or even years. In this manner, information is obtained concerning the relative and absolute health of the subject's hand(s).

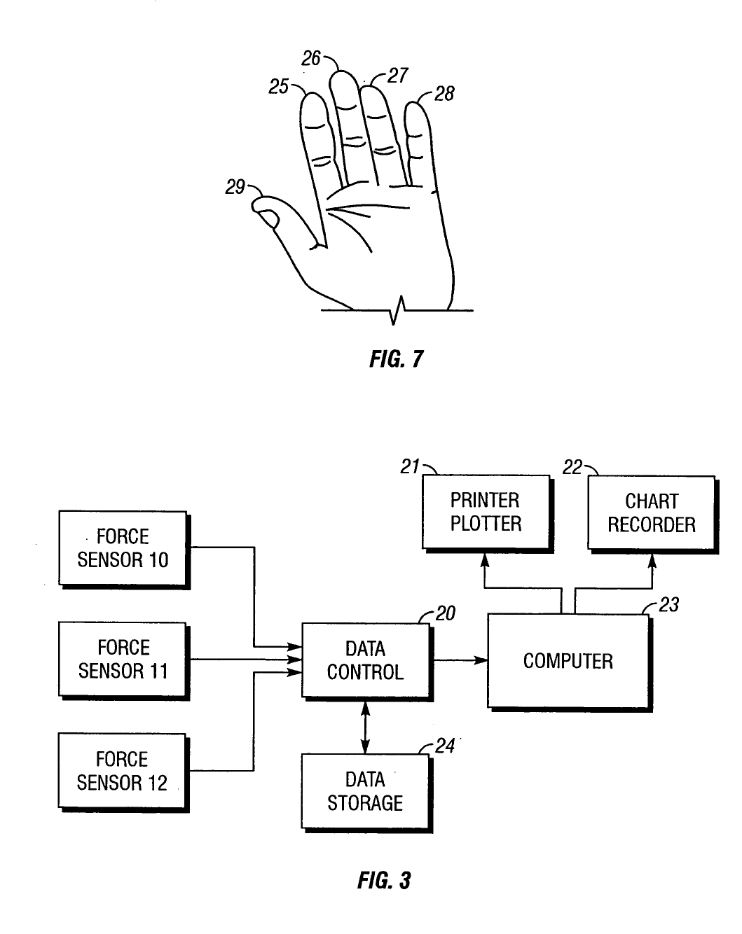

Referring now to FIG. 3, to conduct a test, the subject lightly holds the fixture maintaining the hand-wrist in a prescribed orientation. Upon timing prompts (visual and/or audible) provided by the data control module 20, the test subject squeezes the force sensors, 10, 11, and 12, using only the finger tips. As the test ensues, the data controller 20, collects the data and sends it to storage 24, for later analysis, or to the computer 23 for immediate processing. After processing the data, the results of the test are displayed on the computer 23, printed and/or plotted, 21, 22, and saved to disk 24. The raw data is also available for display on 22 or on the computer 23. Many variations and combinations of regular or irregular squeezes may be studied. Short one or two second squeezes may be repeated for several minutes, or longer squeezes held for five or ten seconds may be repeated for several minutes or a single long pulse of several minutes may be studied. In this manner, hand strength, muscle fatigue, and coordination may be quickly ascertained in a quantifiable, documented manner.

It is also contemplated that disclosed system may provide force prompts. The force prompts alert the user when they have applied a specified force to the fixture or to a particular contact point.

### Strength and Fatigue

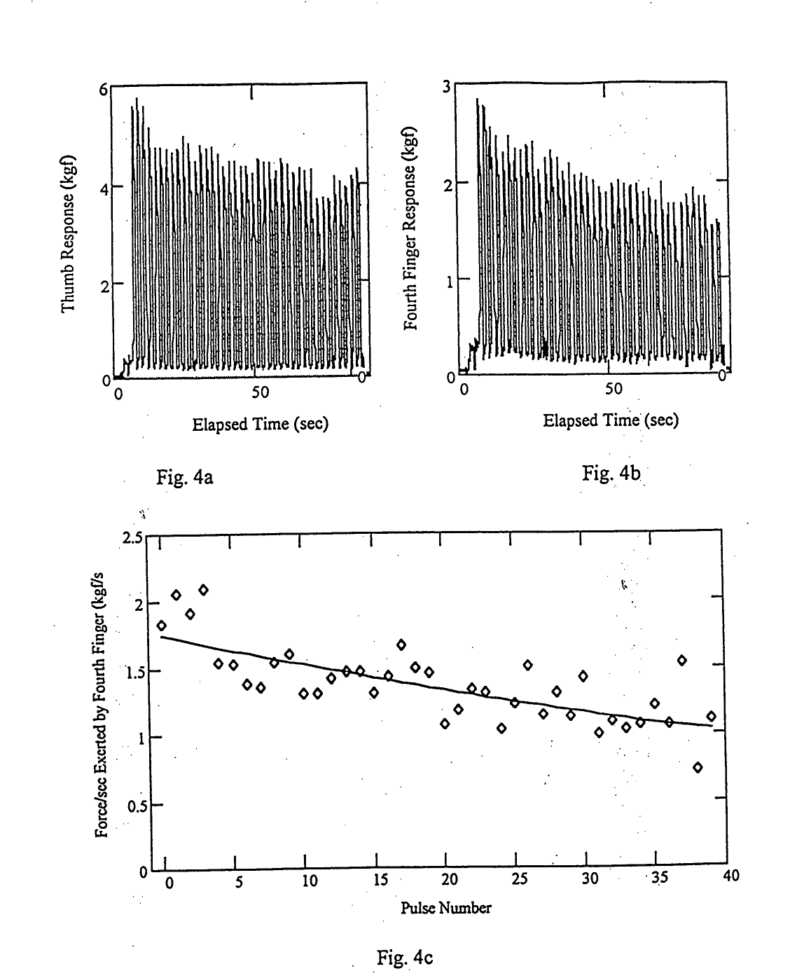

Referring now to FIGS. 4a, 4b and 4c, the applied force data has been collected and displayed for the thumb (FIG. 4a) and the small finger (FIG. 4b) as a function of elapsed time. Both plots show a decrease in the applied force (kgf) as a function of elapsed time (sec). If the short term rate of fatigue is defined as λ, a short term fatigue rate may be computed from the data using Eq. (3):

λ=(1/t) 1n(F/F₀) Eq.(3)

where:

t=elapsed time in seconds

F=Force applied by the digit of interest at a time t

F₀Zero time force intercept, or approximately the initial applied force.

The best manner by which to determine this fatigue rate would be using standard curve fitting techniques, as shown in FIG. 4c, which is a re-plot of the data in FIG. 4b with the pulses integrated and plotted as a function of the pulse number, and in this data set, each pulse represents a time step of 2.31 milliseconds. In this case the term F₀ is the intercept and λ would be the slope determined by a least squares exponential fit to the data. Long term fatigue rates may also be determined in a similar manner. Data collected over many minutes, e.g., 15 or 20 minutes, may be used to numerically or graphically determine a long term fatigue rate in like manner. To date, studies have shown that for compliant subjects, the short term fatigue rates collected over 40 seconds are quite similar if not identical to long term fatigue rates collected over 20 minutes. This similarity thereby provides a subject compliance check throughout the tests.

The absolute and ratiometric strengths of the digits involved in the tests may be determined as time averages or as time dependent parameters which may be determined by computing the ratios of the applied forces. For example, rations, R_{11/10}, R_{12/10}, and R_{11/12} can be computed as shown in Eqs. 4, 5, and 6:

R_{11/10}=(F₁₁/F₁₀) Eq. (4)

R_{12/10}=(F₁₂/F₁₀) Eq. (5)

R_{11/12}=(F₁₁/F₁₂) Eq. (6)

where:

F₁₀=the force applied to contact point 10

F₁₁=the force applied to contact point 11

F₁₂=the force applied to contact point 12.

Comparisons made between the digits of interest as a function of time can also provide the absolute muscle strength tests as in prior art but without the need to compare to a predetermined strength data base. Regardless of the strength of the individual, the ratiometric ratios and the fatigue measurements provide documentation on a specific subject, independent of prior training or abilities. In addition, a non-compliant subject would have to repeat the ratiometric strength results with the appropriate fatigue rates to provide convincing proof of an injury or illness.

### Coordination

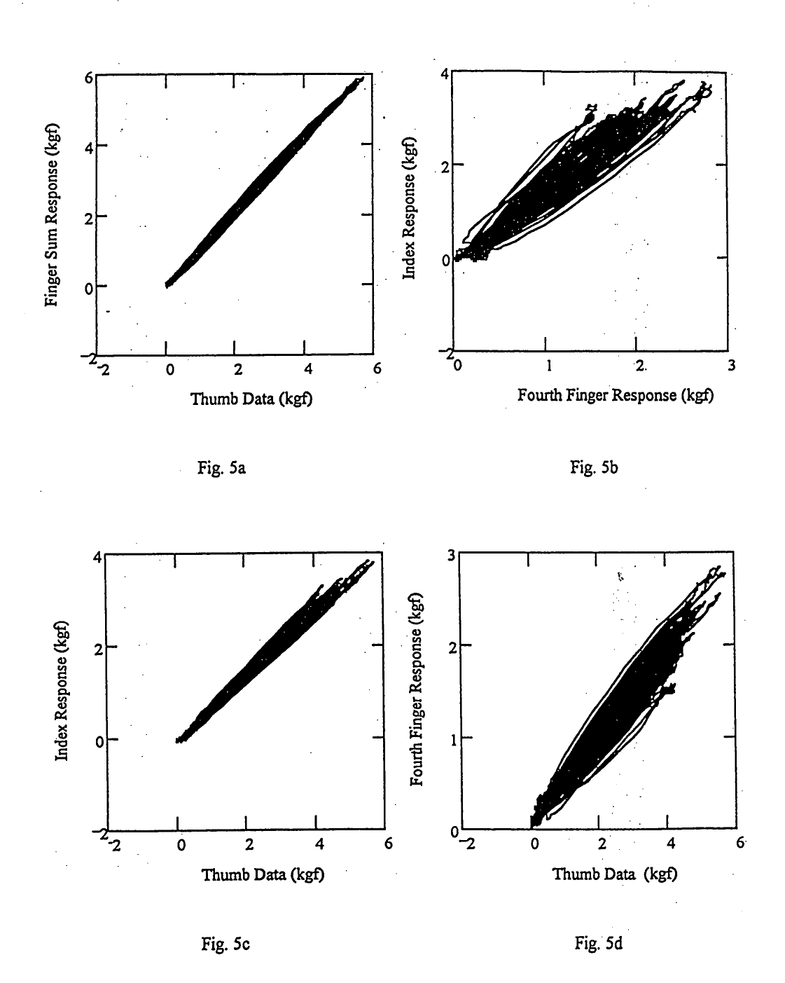

Referring now to FIGS. 5a through 5d, comparisons are being made between the applied forces applied by the thumb, index and small fingers. The force applied by each digit is plotted with respect to a different digit. FIG. 5a shows a quality data check plot. In this plot, the sum of the forces applied by the index and small fingers is plotted against the force applied by the thumb. The Pearson's correlation coefficient for these data is r=0.9996 for the 7132 data points used in this test, indicating a good quality test was completed. FIG. 5b shows the crossplot of the forces applied by the index finger (median nerve) versus the small finger (ulnar nerve). The correlation coefficient is r=0.9820 with n=7132. The crossplot between the thumb and the index finger is shown in FIG. 5c with r=0.9984 and the small finger versus thumb data is shown in FIG. 5d where r=0.9904. The data collected in FIGS. 5a through 5d were collected with the hand-wrist in the neutral position from a test subject with no known neural damage to the hand, but trauma injury was known to have occurred in the past due to a sports injury. Note the increased spread in the data collected for the small finger when plotted against the thumb and index finger. At the same time, the data between the index finger and thumb is quite narrow. This method of presenting the data allows the practitioner to discriminate between injury to specific digits. These tests could have been run using the second or third finger if injury was suspected to those digits. The only requirement is that the proper pair of fingers is chosen to ensure different nerve groups are used in the test. When coupled with hand-wrist positional tests, i.e., neutral versus flexed, a powerful diagnostic screen is provided.

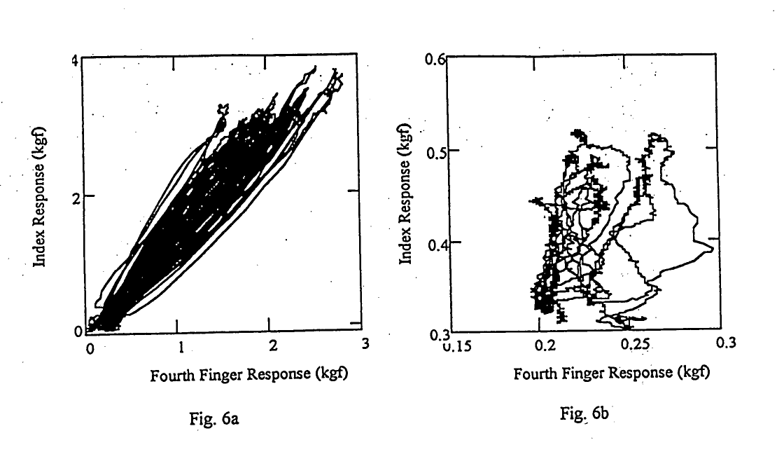

The effectiveness of this screen is shown in FIGS. 6a and 6b. In this case, the data is shown for the index versus small finger for the subject in FIG. 5b versus an individual with a known history of CTS. The correlation coefficient for FIG. 6b is r=0.4629, n=5200. Several important points are evident from these crossplots. The subject hand shown in FIG. 6b is weak, very erratic and poorly coordinated. Even without further testing, a problem is evident.

The method presented provides an indication as to the ratiometric strengths of the digits and as to the relative coordination abilities present between the digits of the hand. Compliance would not be an issue during these tests. Attempts by several individuals with no history of CTS to duplicate the results shown in FIG. 6b have failed to date. This is due to the natural coordination present in a properly functioning hand. Attempts to “jitter” the data by rapidly changing the applied forces fail because the finger movements are still coordinated on the measurement time scale. Only when the coordination is impaired by poor neural feedback will the data exhibit the erratic behavior shown in the figure.

The system and methods of the present invention are not limited to application to individuals afflicted with CTS. Any disease or injury of the hand may be evaluated by these methods. Since different nerve groups are studied, differential results indicate specific problems. For example, strength and fatigue rates of Multiple Sclerosis patients may be evaluated by these methods, providing documentation as a function of time. In fact, the time and/or frequency related outputs provided by the system of the present invention may be studied and used as diagnosis of specific hand, wrist, arm diseases using pattern recognition, neural networks, frequency analysis, signature analysis, plotting or graphic displays for visual analysis by trained doctors/medical technicians. The hand, writ, and arm is referred to as the “wrist complex.” The tests described herein may be repeated at hourly, daily, weekly, yearly or other intervals to determine long term effects.

The exemplary embodiment and method of the invention described with reference to the drawings utilizes the thumb, index and small fingers. Measurements are made from all three, the thumb and index finger for the median nerve and the small finger for the ulnar nerve. Other embodiments of the invention may involve the thumb and any of the other hand digits, or any other three contact points requiring muscle control by muscles innervated by different nerves. However, measurements must be made from at least two digits which involve different nerves, e.g. the median and the ulnar nerves.

FIG. 7 shows an index finger (25), middle finger (26), ring finger (27), little finger (28), and thumb (29).

While the forgoing disclosure and description of the invention is illustrative and explanatory thereof, various changes in the method steps as well as the details of the illustrated preferred embodiment may be made without departing from the spirit of the invention. Accordingly, it is intended that the scope of the invention be limited only by the claims which follow.

All patents and publications mentioned in the specification are indicative of the level of those skilled in the art to which the invention pertains. All patents and publications are herein incorporated by reference to the same extent as if each individual publication was specifically and individually indicated to be incorporated by reference.

What is claimed is:

1. A system for non-invasive analysis of a person's wrist complex comprising:
a fixture with three or more contact points, wherein the three or more contact points are positioned such that forces applied thereto are balanced and wherein such contact points are configured to minimize the side loads.

2. The system of claim 1 wherein force detectors are operatively connected to at least two of said contact points.

3. The system of claim 2 wherein one force detector is engaged by a digit innervated by the ulnar nerve and another force detector is engaged by a digit innervated by the median nerve.

4. The system of claim 3 wherein a display unit displays information about the forces.

5. The system of claim 4 wherein at least one of the contact points is slideably engaged to the fixture.

6. The system of claim 4 wherein at least one of the contact points is slick.

7. The system of claim 6 wherein said contact points are adjustable.

8. The system of claim 6 wherein said contact points are of a rigid, substantially incompressible material.

9. The system of claim 4 wherein said display unit displays information on the forces applied to the force detectors as a function of time.

10. The system of claim 4 wherein the fixture includes contact points for a thumb, index finger, and small finger of a hand.

11. The system of claim 10 wherein said contact points are slick so that the surface of each contact point is generally perpendicular to the force exerted thereon.

12. The system of claim 10 wherein said contact points are slideably engaged to the fixture so that the surface of each contact point is generally perpendicular to the force exerted thereon.

13. The system of claim 10 wherein the projection of forces applied to said contact points do not intersect.

14. The system of claim 10 wherein force detectors are operatively connected to the contact points.

15. The system of claim 4 wherein said fixture has an upper contact point and a lower contact point and the projection of the force applied to the upper contact point projects away from the lower contact point.

16. The system of claim 4 further comprising a displacement measuring means operatively connected to the three or more contact points for measuring the displacement of said three or more contact points.

17. A method of determining the presence or absence of neural, muscular, soft tissue, bone or joint damage to the wrist complex comprising the steps of:
(a) engaging contact points with at least one digit innervated by the ulnar nerve and at least one digit innervated by the median nerve, wherein the contact points are configured to transmit forces normal to their surface;
(b) applying force on the contact points with said digits; and
(c) measuring the force applied to at least two of said contact points to provide quantifiable outputs therefor, wherein the outputs are used to diagnose wrist complex diseases and injuries.

18. The method of claim 17 wherein the quantifiable outputs representing the forces applied by at least two digits innervated by different nerves are displayed.

19. The method of claim 18 wherein the outputs are displayed as a function selected from the group consisting of time, frequency, phase, and any combination thereof.

20. The method of claim 19 wherein the measurements are processed by computer for storage or immediate use.

21. The method of claim 20 wherein the diseases and injuries are diagnosed using a technique selected from the group consisting of pattern recognition, neural networks, frequency analysis, phase analysis, signature analysis, graphic displays, and any combination thereof.

22. The method of claim 20 wherein the measurements are compared to earlier measurements at a frequency selected from the group consisting of hourly, daily, weekly, yearly and any combination thereof to determine long term effects of said diseases or injuries.

23. The method of claim 18 wherein the force is applied to said contact points for a prolonged period of time.

24. The method of claim 18 wherein the force is applied repeatedly to said contact points.

25. The method of claim 18 wherein a visual or audible signal is produced when force should be applied to said contact points.

26. The method of claim 17 wherein said contact points are provided on said fixture that allow displacement measurements to be made.

27. The method of claim 26 wherein said fixture is designed so that the force applied normal to the surface of the contact points is at least 70% of the total force.

28. A force measuring device for transmitting forces applied by a hand comprising
a fixture sized to be gripped by a hand;
at least two contact points operably attached to said fixture to transmit the force applied normal to the surface of said contact point;
a force detector coupled to said contact points for measuring force transmitted to said contact point.

29. The device of claim 28 wherein the fixture comprises an upper surface and a lower surface.

30. The device of claim 28 wherein the upper surface is parallel to the lower surface.

31. The device of claim 28 wherein a contact point is slideably engaged to the upper surface of the fixture.

32. The device of claim 28 wherein a contact point is slideably engaged to said fixture using rollers, wheels, bearings, rails, lubricant or any combination thereof.

33. The device of claim 28 wherein a contact point is slick.

34. The device of claim 28 wherein a contact point is coated with a lubricant.

35. The device of claim 28 wherein said fixture comprises an interior upper surface and an interior lower surface.

36. The device of claim 35 wherein the interior upper surface is parallel to the interior lower surface.

37. The device of claim 35 wherein a contact point is slideably engaged to the interior upper surface of said fixture.

38. The device of claim 35 wherein a contact point is slideably engaged to said fixture using rollers, wheels, bearings, rails, lubricant or any combination thereof.

39. The device of claim 35 wherein a contact point is substantially frictionless to reduce side loads.
